(19) United States
(12) Patent Application Publication
(10) Pub. No.: US 2017/0251915 A1
(43) Pub. Date: Sep. 7, 2017

(54) ENDOSCOPE APPARATUS

(71) Applicant: OLYMPUS CORPORATION, Tokyo (JP)

(72) Inventor: Jumpei TAKAHASHI, Tokyo (JP)

(73) Assignee: OLYMPUS CORPORATION, Tokyo (JP)

(21) Appl. No.: 15/599,666

(22) Filed: May 19, 2017

Related U.S. Application Data
(63) Continuation of application No. PCT/JP2014/081646, filed on Nov. 28, 2014.

(51) Int. Cl. A61B 1/06; A61B 1/00; A61B 1/04

(52) U.S. Cl. CPC A61B 1/0653; A61B 1/04; A61B 1/00009; A61B 1/00059; A61B 1/00186; A61B 1/05

(57) ABSTRACT

An endoscope includes: a light source for emitting white light or narrow band light; a color filter having a first filter for passing light in WLI and NBI, a second filter for passing light in WLI, a third filter for passing light in NBI; and a demosaicing processor that: generates a color image signal based on a luminance component in WLI under the white light; and performs interpolation for a pixel of a luminance component in WLI at a position of a pixel corresponding to the first filter using a pixel corresponding to the second filter under the narrow band light, and then, performs interpolation for a pixel of a luminance component in NBI at the position of the pixel corresponding to the first filter based on a pixel corresponding to the first filter and the pixel of the luminance component in WLI, thereby generating a color image signal.

### CROSS REFERENCES TO RELATED APPLICATIONS

This application is a continuation of PCT international application Ser. No. PCT/JP2014/081646, filed on Nov. 28, 2014 which designates the United States, incorporated herein by reference.

### BACKGROUND

1. Technical Field

The disclosure relates to an endoscope apparatus configured to be introduced into a living body to acquire images in the living body.

2. Related Art

Conventionally, endoscope apparatuses have been widely used for various kinds of examinations in a medical field and an industrial field. Among such endoscope apparatuses, a medical endoscope apparatus is capable of acquiring an image inside a body cavity even without cutting a subject by inserting a flexible insertion portion, which is provided with an image sensor including a plurality of pixels at a distal end thereof and formed in an elongated shape, into the body cavity of the subject such as a patient. Thus, the medical endoscope apparatus imposes a light burden on the subject, and has been widely used.

As an observation method of such an endoscope apparatus, a white light imaging (WLI) method using white illumination light (white illumination light) and a narrow band imaging (NBI) method using illumination light (narrow band illumination light) formed of two kinds of narrow band light included in blue and green wavelength bands, respectively, have been already widely known in this technical field. In regard to these observation methods of the endoscope apparatus, there is a demand to perform observation by switching between the white illumination light imaging method (a WLI mode) and the narrow band imaging method (an NBI mode).

A color filter in which a plurality of filters are arranged in a matrix is provided on a light receiving surface of an image sensor to allow acquisition of an image captured by a single-plate image sensor in order to generate and display a color image in the above-described observation methods. In general, known is a color filter in which a plurality of filters are arrayed in a matrix using a filter array called a Bayer array, as a unit, which is formed by arraying filters (hereinafter, referred to as primary color filters) that pass light having wavelength bands of red (R), green (G), and blue (B), which are primary colors, in 2×2 matrix. Each pixel receives light of a wavelength band having passed through the filter, and the mage sensor generates an electric signal of a color component corresponding to the light of the wavelength band.

In this regard, disclosed is an image sensor provided with a color filter in which a plurality of filters are arrayed in a matrix using four filters, as a unit, selected from a G filter that passes light of the green wavelength band and filters (hereinafter, referred to as complementary color filters) that pass light of wavelength bands of complementary colors, such as yellow (Ye) and cyan (Cy) (for example, see JP 2003-87804 A). The wavelength bands of light that passes through the complementary color filter have a wider range as compared to the wavelength bands of light that passes through the primary color filter, and thus, higher sensitivity is obtained and noise can be suppressed in the case of using the complementary color filter than the case of using the primary color filter.

In the WLI mode, a signal of a green component with which a blood vessel and a ductal structure of the living body are clearly displayed, that is, a signal (G signal) acquired using a G pixel (a pixel in which the G filter is arranged, similar definition is applied for an R pixel, a B pixel, a Ye pixel, and a Cy pixel) has the highest degree of contribution to luminance of an image. In the NBI mode, on the other hand, a signal of a blue component with which a blood vessel and a ductal structure of a living body surface layer are clearly displayed, that is, a signal (B signal) acquired using the B pixel has the highest degree of contribution to the luminance of the image.

### SUMMARY

In some embodiments, provided an endoscope apparatus for performing white illumination light imaging and narrow band illumination light imaging. The endoscope apparatus includes: a light source unit configured to emit one of white illumination light and narrow band illumination light, the white illumination light including red wavelength band light, green wavelength band light, and blue wavelength band light, the narrow band illumination light including light of two narrow bands included in a wavelength band of a luminance component in the white illumination light imaging and a wavelength band of a luminance component in the narrow band illumination light imaging; an image sensor including a plurality of pixels arranged in matrix and configured to perform photoelectric conversion on light received by each of the plurality of pixels to generate an electric signal; a color filter arranged on a light receiving surface of the image sensor and formed by arraying a plurality of filter units, each of the plurality of filter units including a first filter, a second filter, and a third filter, the first filter being configured to pass light of the wavelength band of the luminance component in the white illumination light imaging and the wavelength band of the luminance component in the narrow band illumination light imaging, the second filter being configured to pass light of the wavelength band of the luminance component in the white illumination light imaging, and the third filter being configured to pass light of the wavelength band of the luminance component in the narrow band illumination light imaging; and a demosaicing processing unit configured to: generate a color image signal including a plurality of color components based on the luminance component in the white illumination light imaging when the light source unit emits the white illumination light; and perform interpolation for a pixel value of the luminance component in the white illumination light imaging at a position of a pixel corresponding to the first filter using a pixel value of a pixel corresponding to the second filter when the light source unit emits the narrow band illumination light, and then, perform interpolation for a pixel value of the luminance component in the narrow band illumination light imaging at the position of the pixel corresponding to the first filter based on a pixel value of the pixel corresponding to the first filter and the pixel value of the luminance component in the white illumination light imaging obtained by the interpolation, thereby generating a color image signal including a plurality of color components.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a diagram illustrating a schematic configuration of an endoscope apparatus according to an embodiment of the present invention;

FIG. 2 is a schematic diagram illustrating a schematic configuration of the endoscope apparatus according to the embodiment of the present invention;

FIG. 3 is a schematic diagram illustrating a configuration of a pixel according to the embodiment of the present invention;

FIG. 4 is a schematic diagram illustrating an exemplary configuration of a color filter according to the embodiment of the present invention;

FIG. 5 is a graph illustrating exemplary characteristics of a G filter of the color filter according to the embodiment of the present invention and is a graph illustrating a relationship between a wavelength of light and transmittance of each filter;

FIG. 6 is a graph illustrating exemplary characteristics of an Mg filter of the color filter according to the embodiment of the present invention and is a graph illustrating a relationship between a wavelength of light and transmittance of each filter;

FIG. 7 is a graph illustrating exemplary characteristics of a Cy filter of the color filter according to the embodiment of the present invention and is a graph illustrating a relationship between a wavelength of light and transmittance of each filter;

FIG. 8 is a graph illustrating exemplary characteristics of a Ye filter in the color filter according to the embodiment of the present invention and is a graph illustrating a relationship between a wavelength of light and transmittance of each filter;

FIG. 9 is a graph illustrating a relationship between a wavelength and the amount of light of illumination light emitted from an illumination unit of the endoscope apparatus according to the embodiment of the present invention;

FIG. 10 is a graph illustrating a relationship between a wavelength and transmittance of illumination light from a switching filter included in the illumination unit of the endoscope apparatus according to the embodiment of the present invention;

FIG. 11 is a configuration of the color filter according to the embodiment of the present invention and is a diagram for describing a function of a pixel in an NBI mode;

FIG. 12 is a flowchart illustrating demosaicing processing in the NBI mode which is performed by a demosaicing processing unit of the endoscope apparatus according to the embodiment of the present invention;

FIG. 13 is a flowchart illustrating demosaicing processing in a WLI mode which is performed by a demosaicing processing unit of the endoscope apparatus according to the embodiment of the present invention;

FIG. 14 is a flowchart illustrating signal processing which is performed by a processor of the endoscope apparatus according to the embodiment of the present invention;

FIG. 15 is a schematic diagram illustrating a configuration of a color filter according to a first modified example of the embodiment of the present invention;

FIG. 16 is a configuration of the color filter according to the first modified example of the embodiment of the present invention and is a diagram for describing a function of a pixel in an NBI mode; and

FIG. 17 is a schematic diagram illustrating a configuration of a color filter according to a third modified example of the embodiment of the present invention.

### DETAILED DESCRIPTION

Hereinafter, modes for carrying out the present invention (hereinafter, referred to as “embodiment(s)”) will be described. In the embodiments, reference will be made to a medical endoscope apparatus for capturing and displaying images in a body cavity of a subject such as a patient. The invention is not limited by the embodiments. The same reference signs are used to designate the same elements throughout the drawings.

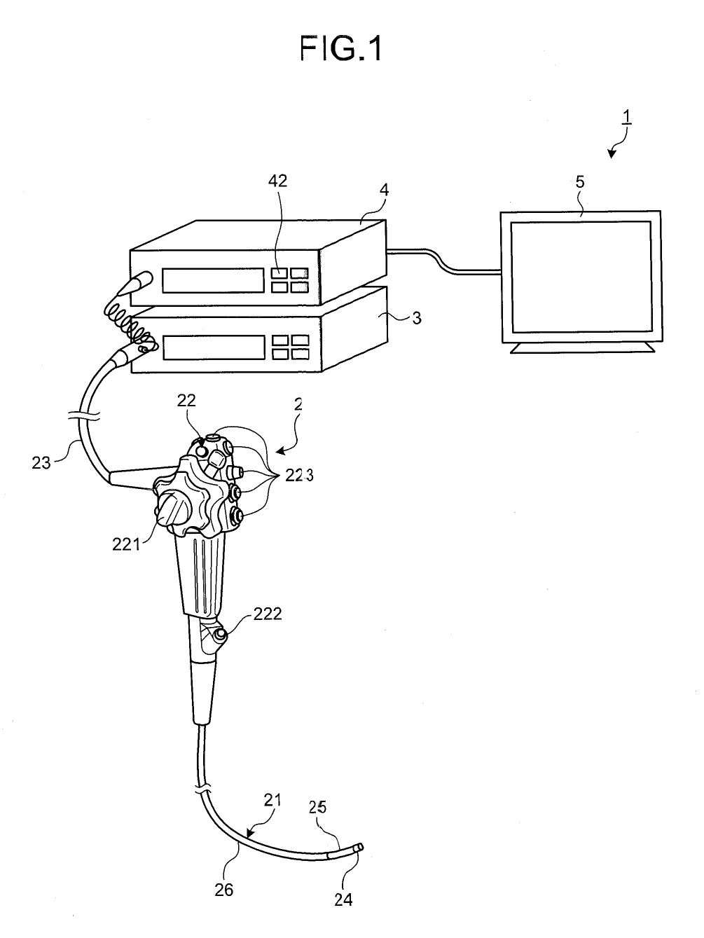

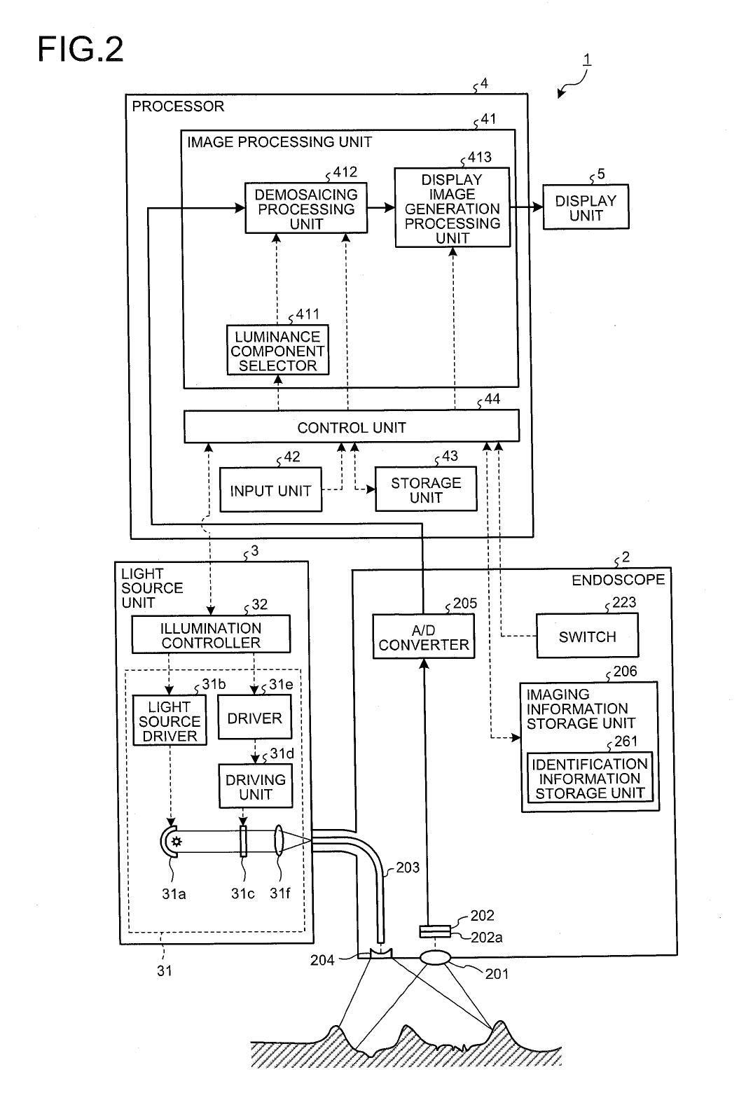

FIG. 1 is a diagram illustrating a schematic configuration of an endoscope apparatus according to an embodiment of the present invention. FIG. 2 is a schematic diagram illustrating a schematic configuration of the endoscope apparatus according to the embodiment. An endoscope apparatus 1 illustrated in FIGS. 1 and 2 includes: an endoscope 2 that captures an in-vivo image of an observed region by inserting an insertion portion 21 into a body cavity of a subject and generates an electric signal; a light source unit 3 that generates illumination light to be emitted from a distal end of the endoscope 2; a processor 4 that performs predetermined image processing on the electric signal acquired by the endoscope 2 and integrally controls the entire operation of the endoscope apparatus 1; and a display unit 5 that displays the in-vivo image having been subjected to the image processing by the processor 4. The endoscope apparatus 1 inserts the insertion portion 21 into the body cavity of the subject such as a patient and acquires the in-vivo image inside the body cavity. A user such as a doctor examines presence or absence of a bleeding site or a tumor site, which is a detection target region, by observing the acquired in-vivo image. An arrow in a solid line indicates transmission of an electric signal relating to the image and an arrow in a broken line indicates transmission of an electric signal relating to control in FIG. 2.

The endoscope 2 includes: the insertion portion 21 that has flexibility and is formed in an elongated shape; an operating unit 22 that is connected to a proximal end side of the insertion portion 21 and receives input of various kinds of operation signals; and a universal cord 23 that extends from the operating unit 22 in a direction different from an extending direction of the insertion portion 21 and includes various types of built-in cables which are connected to the light source unit 3 and the processor 4.

The insertion portion 21 includes: a distal end portion 24 that includes a built-in image sensor 202 in which pixels (photodiodes) to receive light are arrayed in a matrix and which generates an image signal by performing photoelectric conversion with respect to the light received by the pixels; a bending portion 25 that is configured using a plurality of bending pieces and can be freely bent; and an elongated flexible tube portion 26 that is connected to a proximal end side of the bending portion 25 and has flexibility.

The operating unit 22 includes: a bending knob 221 to bend the bending portion 25 in an up-and-down direction and a right-and-left direction; a treatment tool insertion portion 222 to insert a treatment tool such as a living body forceps, an electrical scalpel, or a test probe into the body cavity of the subject; and a plurality of switches 223 to receive a command signal to cause the light source unit 3 to perform a switching operation of illumination light, an operation command signal for an external device that is connected to the treatment tool and the processor 4, a water feed command signal to feed water, a suction command signal to perform suctioning, and the like. The treatment tool to be inserted from the treatment tool insertion portion 222 is exposed from an opening (not illustrated) via a treatment tool channel (not illustrated) which is provided at a distal end of the distal end portion 24. The switch 223 may be configured to include an illumination light changeover switch to switch the illumination light (imaging method) of the light source unit 3.

At least a light guide 203 and a cable assembly formed by assembling one or a plurality of signal lines are built in the universal cord 23. The cable assembly is the signal line to transmit and receive signals among the endoscope 2, the light source unit 3, and the processor 4, and includes a signal line to transmit and receive setting data, a signal line to transmit and receive an image signal, a signal line to transmit and receive a drive timing signal to drive the image sensor 202, and the like.

In addition, the endoscope 2 includes an imaging optical system 201, an image sensor 202, a light guide 203, an illumination lens 204, an A/D converter 205, and an imaging information storage unit 206.

The imaging optical system 201 is provided at the distal end portion 24 and collects light at least from an observed region. The imaging optical system 201 is configured using one or a plurality of lenses. The imaging optical system 201 may be provided with an optical zooming mechanism to change a viewing angle and a focusing mechanism to change a focal point.

The image sensor 202 is provided perpendicularly to an optical axis of the imaging optical system 201, performs photoelectric conversion on an image of light formed by the imaging optical system 201, and generates an electric signal (image signal). The image sensor 202 is implemented using a charge coupled device (CCD) image sensor, a complementary metal oxide semiconductor (CMOS) image sensor, and the like.

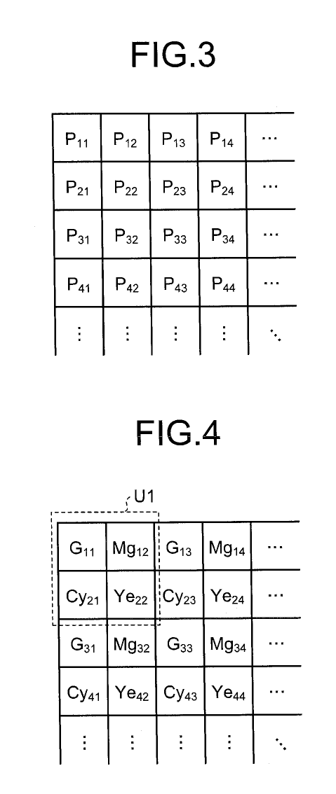

FIG. 3 is a schematic diagram illustrating a configuration of a pixel of the image sensor according to the embodiment. The image sensor 202 includes a plurality of pixels configured to receive light from the imaging optical system 201, and the plurality of pixels are arrayed in a matrix. Further, the image sensor 202 generates an imaging signal formed using an electric signal which is generated by performing photoelectric conversion on the light received by each pixel. This imaging signal includes a pixel value (luminance value) of each pixel and position information of pixels, and the like. In FIG. 3, a pixel which is arranged at i-th row and j-th column is denoted as a pixel Pᵢⱼ.

The image sensor 202 is provided with a color filter 202a that is provided between the imaging optical system 201 and the image sensor 202 and includes a plurality of filters each of which is configured to pass light having individually set wavelength bands. The color filter 202a is provided on a light receiving surface of the image sensor 202.

FIG. 4 is a schematic diagram illustrating an exemplary configuration of the color filter according to the embodiment. The color filter 202a according to the embodiment is configured by arranging filter units U1, formed using four filters arrayed in a 2×2 matrix, to be arrayed in a matrix in accordance with arrangement of the pixels Pᵢⱼ. In other words, the color filter 202a is configured by arranging basic patterns repeatedly, using a filter array of the filter units U1 as the basic pattern. The single filter, which passes light of a predetermined wavelength band, is arranged on each light receiving surface of the respective pixels. Thus, the pixel Pᵢⱼ, provided with the filter, receives light of a wavelength band that pass through the corresponding filter. For example, the pixel Pᵢⱼ that is provided with the filter that passes light of a green (G) wavelength band receives light of the green wavelength band. Hereinafter, the pixel Pᵢⱼ that receives light of the green wavelength band is referred to as a G pixel. Similarly, the pixel that receives light of a magenta (Mg) wavelength band is referred to as an Mg pixel, the pixel that receives light of a cyan (Cy) wavelength band is referred to as a Cy pixel, and the pixel that receives light of a yellow (Ye) wavelength band is referred to as a Ye pixel.

Herein, the filter unit U1 passes light of a blue (B) wavelength band H_{B}, a green wavelength band H_{G}, and a red (R) wavelength band H_{R}. In addition, the filter unit U1 according to the embodiment is configured using a green filter (G filter) that passes light of the wavelength band H_{G}, a magenta filter (Mg filter) that passes light of the wavelength band H_{B} and the wavelength band H_{R}, a cyan filter (Cy filter) that passes light of the wavelength band H_{B} and the wavelength band H_{G}, and a yellow filter (Ye filter) that passes light of the wavelength band H_{G} and the wavelength band H_{R} as illustrated in FIG. 4. For example, the blue, green, and red wavelength bands H_{B}, H_{G}, and H_{R} are given such that, for example, the wavelength band H_{B} is 400 nm to 500 nm, the wavelength band H_{G} is 480 nm to 600 nm, and the wavelength band H_{R} is 580 nm to 700 nm. Hereinafter, when the G filter is provided at a position corresponding to the pixel Pᵢⱼ, this G filter is denoted as Gᵢⱼ. Similarly, the Mg filter is denoted as Mgᵢⱼ when being provided at a position corresponding to the pixel Pᵢₖ, the Cy filter is denoted as Cyᵢⱼ when being provided at a position corresponding to the pixel Pᵢⱼ, and the Ye filter is denoted as Yeᵢⱼ when being provided at a position corresponding to the pixel Pᵢⱼ.

FIGS. 5 to 8 are graphs illustrating exemplary characteristics of each filter of the color filter according to the embodiment and are graphs illustrating a relationship between a wavelength of light and transmittance of each filter. In the respective drawings, transmittance curves are normalized so that the maximum value of the transmittance of each filter may be identical. FIG. 5 is a graph illustrating exemplary characteristics of the G filter of the color filter according to the embodiment and illustrating a relationship between the wavelength of light and the transmittance of each filter. A curved line L_{g} illustrated in FIG. 5 represents the transmittance curve of the G filter. As illustrated in FIG. 5, the G filter passes light of the wavelength band H_{G}.

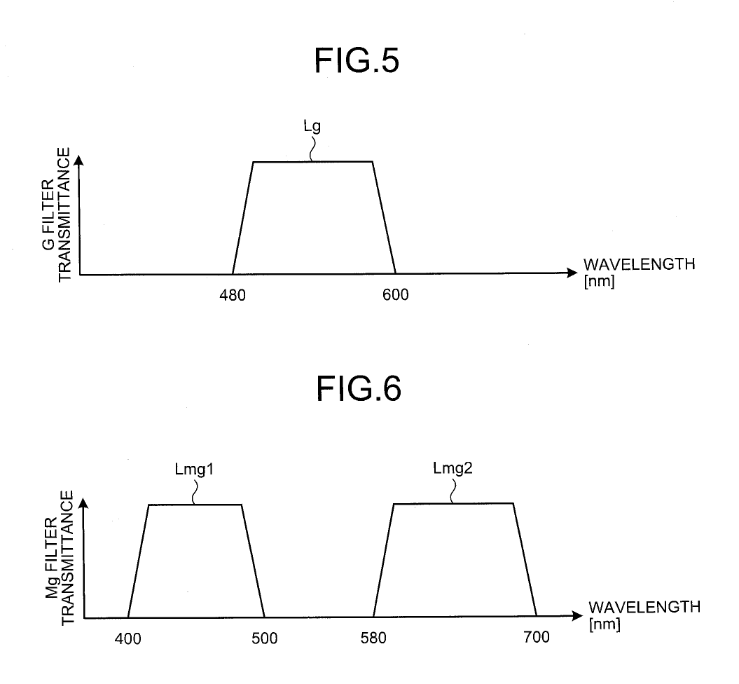

FIG. 6 is a graph illustrating exemplary characteristics of the Mg filter of the color filter according to the embodiment and illustrating a relationship between the wavelength of light and the transmittance of each filter. Curved lines L_{mg}1 and L_{mg}2 illustrated in FIG. 6 represent the transmittance curves of the Mg filter. As illustrated in FIG. 6, the Mg filter passes light of the wavelength band H_{B} and the wavelength band H_{R}.

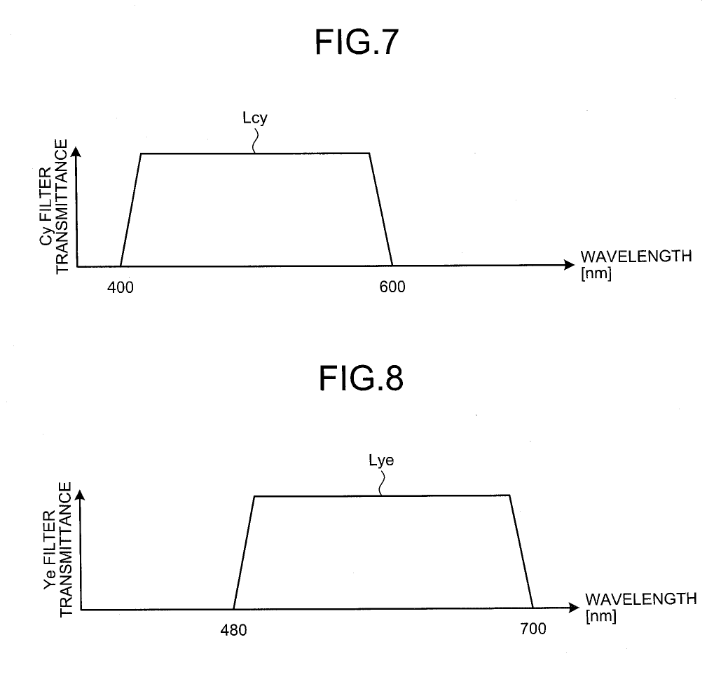

FIG. 7 is a graph illustrating exemplary characteristics of the Cy filter of the color filter according to the embodiment and illustrating a relationship between the wavelength of light and the transmittance of each filter. A curved line L_{cy} illustrated in FIG. 7 represents the transmittance curve of the Cy filter. As illustrated in FIG. 7, the Cy filter passes light of the wavelength band H_{B} and the wavelength band H_{G}.

FIG. 8 is a graph illustrating exemplary characteristics of the Ye filter of the color filter according to the embodiment and illustrating a relationship between the wavelength of light and the transmittance of each filter. A curved line L_{ye} illustrated in FIG. 8 represents the transmittance curve of the Ye filter. As illustrated in FIG. 8, the Ye filter passes light of the wavelength band H_{G} and the wavelength band H_{R}.

Returning to the explanation of FIGS. 1 and 2, the light guide 203 is configured using a glass fiber or the like and forms a light guide path of light emitted from the light source unit 3.

The illumination lens 204 is provided at a distal end of the light guide 203, diffuses light guided by the light guide 203, and emits the light to the outside of the distal end portion 24.

The A/D converter 205 performs A/D conversion on the imaging signal generated by the image sensor 202 and outputs this converted imaging signal to the processor 4.

The imaging information storage unit 206 stores various programs configured to operate the endoscope 2 and data including various parameters necessary for operations of the endoscope 2, identification information of the endoscope 2, and the like. In addition, the imaging information storage unit 206 includes an identification information storage unit 261 that stores the identification information. The identification information includes unique information (ID) of the endoscope 2, a model year, specification information, a transmission method, filter array information relating to the color filter 202a, and the like. The imaging information storage unit 206 is implemented using a flash memory or the like.

Next, a configuration of the light source unit 3 will be described. The light source unit 3 includes an illumination unit 31 and an illumination controller 32.

The illumination unit 31 switches and emits a plurality of beams of illumination light whose wavelength bands are different from each other under control of the illumination controller 32. The illumination unit 31 includes a light source 31a, a light source driver 31b, a switching filter 31c, a driving unit 31d, a driver 31e, and a condenser lens 31f.

The light source 31a emits white illumination light including light of red, green, and blue wavelength bands H_{R}, H_{G}, and H_{B} under the control of the illumination controller 32. The white illumination light generated by the light source 31a passes through the switching filter 31c, the condenser lens 31f, and the light guide 203, and then, is emitted to the outside from the distal end portion 24. The light source 31a is implemented using a light source, such as a white LED and a xenon lamp that emits white light.

The light source driver 31b causes the light source 31a to emit the white illumination light by supplying a current to the light source 31a under the control of the illumination controller 32.

The switching filter 31c passes only blue narrow band light and green narrow band light out of the white illumination light emitted by the light source 31a. The switching filter 31c is arranged on an optical path of the white illumination light, emitted from the light source 31a, to be removable under the control of the illumination controller 32. The switching filter 31c is arranged on the optical path of the white illumination light to pass only beams of light having two narrow bands. To be specific, the switching filter 31c passes narrow band illumination light including light of a narrow band T_{B} (for example, 400 nm to 445 nm) included in the wavelength band H_{B}, and light of a narrow band T_{G} (for example, 530 nm to 550 nm) included in the wavelength band H_{G}. The narrow bands T_{B} and T_{G} are wavelength bands of blue light and green light, respectively, which are easily absorbed by hemoglobin in blood. At least a wavelength band of 405 nm to 425 nm may be included as the narrow band T_{B}. The light that is emitted in the state of being limited to such a band is referred to as the narrow band illumination light, and observation of an image using this narrow band illumination light is referred to as a narrow band imaging (NBI) method.

The driving unit 31d is configured using a stepping motor, a DC motor, or the like and causes the switching filter 31c to be inserted into or removed from the optical path of the light source 31a.

The driver 31e supplies a predetermined current to the driving unit 31d under the control of the illumination controller 32.

The condenser lens 31f collects the white illumination light emitted from the light source 31a or the narrow band illumination light that has passed through the switching filter 31c, and emits the collected light to the outside (light guide 203) of the light source unit 3.

The illumination controller 32 controls the light source driver 31b to cause the on/off operation of the light source 31a, and controls the driver 31e to cause the switching filter 31c to be inserted into or removed from the optical path of the light source 31a, thereby controlling types (bands) of the illumination light to be emitted from the illumination unit 31.

To be specific, the illumination controller 32 causes the switching filter 31c to be inserted into or removed from the optical path of the light source 31a so as to control switching of the illumination light emitted from the illumination unit 31 to any light between the white illumination light and the narrow band illumination light. In other words, the illumination controller 32 performs switching to any imaging method between the white light imaging (WLI) method using the white illumination light including light of the wavelength bands H_{B}, H_{G}, and H_{R}, and the narrow band imaging (NBI) method using the narrow band illumination light including light of the narrow bands T_{B} and T_{G}.

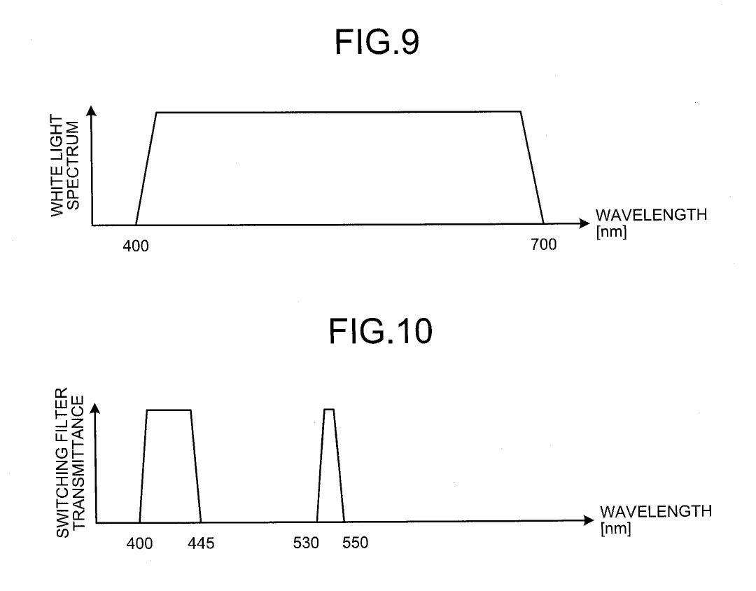

FIG. 9 is a graph illustrating a relationship between a wavelength and the amount of light of the illumination light emitted from the illumination unit of the endoscope apparatus according to the embodiment. FIG. 10 is a graph illustrating a relationship between a wavelength and transmittance of the illumination light from the switching filter included in the illumination unit of the endoscope apparatus according to the embodiment. When the switching filter 31c is removed from the optical path of the light source 31a under the control of the illumination controller 32, the illumination unit 31 emits the white illumination light including light of the wavelength bands H_{B}, H_{G} and H_{R} (see FIG. 9). On the contrary, when the switching filter 31c is inserted into the optical path of the light source 31a under the control of the illumination controller 32, the illumination unit 31 emits the narrow band illumination light including light of the narrow bands T_{B} and T_{G} (see FIG. 10).

The illumination unit 31 has been described as the unit of switching the illumination light, emitted from the illumination unit 31 depending on the insertion or removal of the switching filter 31c, to any light between the white illumination light and the narrow band illumination light, with respect to the white illumination light emitted from the light source 31a. However, the illumination unit 31 may be configured to use a rotation filter, to emit any light between the white illumination light and the narrow band illumination light by switching between two light sources (for example, a LED light source and a laser light source) which emit the white illumination light and the narrow band illumination light, respectively, or to include a plurality of light sources each of which emits the narrow band illumination light and control the light sources to emit the white illumination light by combining the plurality of light sources at the time of white illumination light imaging.

Next, a configuration of the processor 4 will be described. The processor 4 includes an image processing unit 41, an input unit 42, a storage unit 43, and a control unit 44.

The image processing unit 41 performs predetermined image processing based on the imaging signal from the endoscope 2 (A/D converter 205) to generate a display image signal for display of the display unit 5. The image processing unit 41 includes a luminance component selector 411, a demosaicing processing unit 412, and a display image generation processing unit 413.

The luminance component selector 411 determines a switching operation of the illumination light performed by the illumination controller 32, that is, determines which of the white illumination light and the narrow band illumination light the illumination light emitted by the illumination unit 31 is. The luminance component selector 411 selects a luminance component (pixel that receives light of the luminance component) to be used by the demosaicing processing unit 412 according to the determined illumination light. For example, a relative luminosity factor of a human eye is the highest in the white illumination light imaging, and a green component with which a blood vessel and a ductal structure of the living body are clearly displayed becomes the luminance component. On the other hand, the luminance component to be selected differs depending on a subject in the narrow band illumination light imaging. Therefore, there is a case where the green component is selected similarly to the white illumination light imaging, and also, there is a case where the luminance component is different from that of the white light imaging. To be specific, the above-described NBI observation is a representative example of a case where a blue component or a red component becomes the luminance component in the narrow band imaging. In this case, the blue component with which a blood vessel and a ductal structure of a living body surface layer are clearly displayed becomes the luminance component. In the embodiment, the green component is set as the luminance component in the white illumination light imaging, and the blue component is set as the luminance component in the narrow band imaging. The luminance component is automatically set by determining an imaging method as long as the luminance component is set for each imaging method in advance, and thus, it is possible to omit the selection process performed by the luminance component selector 411.

Since the NBI observation is used as the narrow band illumination light imaging in the embodiment, the Cy filter corresponds to a first filter that passes light of the wavelength bands (wavelength bands H_{B} and H_{G}) of the respective luminance components in the white illumination light imaging and the narrow band illumination light imaging, the G filter and the Ye filter correspond to second filters that pass light of the wavelength band (wavelength band H_{G}) of the luminance component (green component) in the white illumination light imaging and block light of the wavelength band of the luminance component (blue component) in the narrow band illumination light imaging, and the Mg filter corresponds to a third filter that passes light of the wavelength band (the wavelength band H_{B}) of the luminance component in the narrow band illumination light imaging and blocks light of the wavelength band of the luminance component in the white illumination light imaging.

The demosaicing processing unit 412 generates a color image signal by discriminating an interpolation direction from a correlation of color information (pixel values) of the plurality of pixels based on the imaging signal from the endoscope 2 (A/D converter 205) and performing interpolation based on the color information of the pixels arrayed in the discriminated interpolation direction. The demosaicing processing unit 412 generates the color image signal by performing interpolation processing on a luminance component based on a pixel of the luminance component selected by the luminance component selector 411 (hereinafter, referred to as a selected pixel), and then, performing interpolation processing on color components other than the luminance component.

The display image generation processing unit 413 executes gradation conversion, magnification processing, enhancement processing for the blood vessel and the ductal structure of the living body, and the like with respect to the electric signal generated by the demosaicing processing unit 412. After executing the predetermined processing, the display image generation processing unit 413 outputs the processed electric signal to the display unit 5 as a display image signal for display.

The image processing unit 41 performs OB clamp processing, gain adjustment processing, and the like in addition to the above-described demosaicing processing. In the OB clamp processing, the image processing unit 41 executes processing for correcting an offset amount of a black level with respect to the electric signal input from the endoscope 2 (A/D converter 205). In the gain adjustment processing, the image processing unit 41 executes adjustment processing of a brightness level with respect to the image signal having been subjected to the demosaicing processing.

The input unit 42 is an interface configured to perform input or the like from a user with respect to the processor 4, and includes a power switch configured to turn on or off a power supply, a mode switching button configured to switch a shooting mode and other various modes, an illumination light switching button configured to switch the illumination light (imaging method) of the light source unit 3, and the like.

The storage unit 43 records various programs configured to operate the endoscope apparatus 1 and data including various parameters and the like necessary for operations of the endoscope apparatus 1. The storage unit 43 may store information relating to the endoscope 2, for example, a relationship table between the unique information (ID) of the endoscope 2 and information relating to filter arrangement of the color filter 202a. The storage unit 43 is implemented using a semiconductor memory such as a flash memory and a dynamic random access memory (DRAM).

The control unit 44 is configured using a CPU and the like, and performs driving control of the respective elements including the endoscope 2 and the light source unit 3 and input and output control of information with respect to the respective elements, and the like. The control unit 44 transmits setting data (for example, pixels to be read and the like) for imaging control, recorded in the storage unit 43, a timing signal relating to imaging timing, and the like to the endoscope 2 via a predetermined signal line. The control unit 44 outputs color filter information (identification information) acquired via the imaging information storage unit 206 to the image processing unit 41, and outputs information on arrangement of the switching filter 31c to the light source unit 3 based on the color filter information.

Next, the display unit 5 will be described. The display unit 5 receives the display image signal generated by the processor 4 via a video cable, and displays an in-vivo image corresponding to the display image signal. The display unit 5 is configured using liquid crystal or organic electro luminescence (EL).

Next, reference will be made to the signal processing performed by each unit of the processor 4 of the endoscope apparatus 1. The luminance component selector 411 determines any imaging method between the white illumination light imaging method and the narrow band imaging method that is used to generate the input imaging signal. To be specific, the luminance component selector 411 determines any imaging method used to generate the input image signal based on a control signal (for example, information on the illumination light or information indicating the imaging method) from the control unit 44, for example.

When determining that the input imaging signal has been generated using the white illumination light imaging method, the luminance component selector 411 selects and sets the G pixel as a selected pixel, and outputs setting information that has been set to the demosaicing processing unit 412. To be specific, the luminance component selector 411 outputs position information of the G pixel, set as the selected pixel based on the identification information (information of the color filter 202a), for example, information on a row and a column of the G pixel.

On the contrary, when determining that the input imaging signal has been generated using the narrow band imaging method, the luminance component selector 411 selects and sets the Mg pixel and the Cy pixel (pixels including a signal of the B component) as a selected pixel, and outputs setting information that has been set to the demosaicing processing unit 412.

Next, the interpolation processing performed by the demosaicing processing unit 412 will be described. The demosaicing processing unit 412 generates the color image signal by discriminating the interpolation direction from the correlation of the color information (pixel values) of the plurality of pixels based on an unsynchronized imaging signal from the endoscope 2 (A/D converter 205) and performing the interpolation based on the color information of the pixels arrayed in the discriminated interpolation direction. The demosaicing processing unit 412 performs different types of signal processing between the NBI mode and the WLI mode.

Demosaicing Processing in NBI Mode

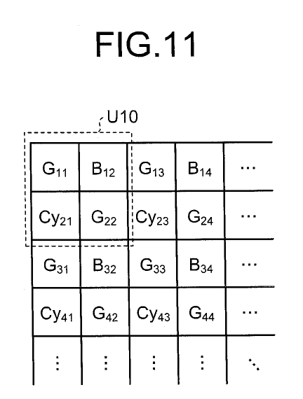

FIG. 11 is a configuration of the color filter according to the embodiment and is a diagram for describing a function of the pixel in the NBI mode. The narrow band illumination light emitted from the light source unit 3 includes light of the narrow bands T_{B} and T_{G} as illustrated in FIG. 10. Thus, the Mg filter passes only light of the narrow band T_{B}, and accordingly, the Mg pixel can be considered to be equivalent to the B pixel. Similarly, the Ye filter passes only light of the narrow band T_{G}, and accordingly, the Ye pixel can be considered to be equivalent to the G pixel. Here, the B pixel corresponds to a pixel provided with a B filter that passes light having a wavelength band of 400 to 480 nm. Accordingly, a complementary color filter array (the filter unit U1) illustrated in FIG. 4 can be considered to be equivalent to a filter array (a filter unit U10) illustrated in FIG. 11 in the NBI mode. In the filter array illustrated in FIG. 11, the filters that pass light of the wavelength band H_{G} are arranged in a checkerboard pattern. In the following demosaicing processing in the NBI mode, the filter array illustrated in FIG. 11 is employed in which the Mg pixel and the Ye pixel are regarded as the B pixel and the G pixel, respectively.

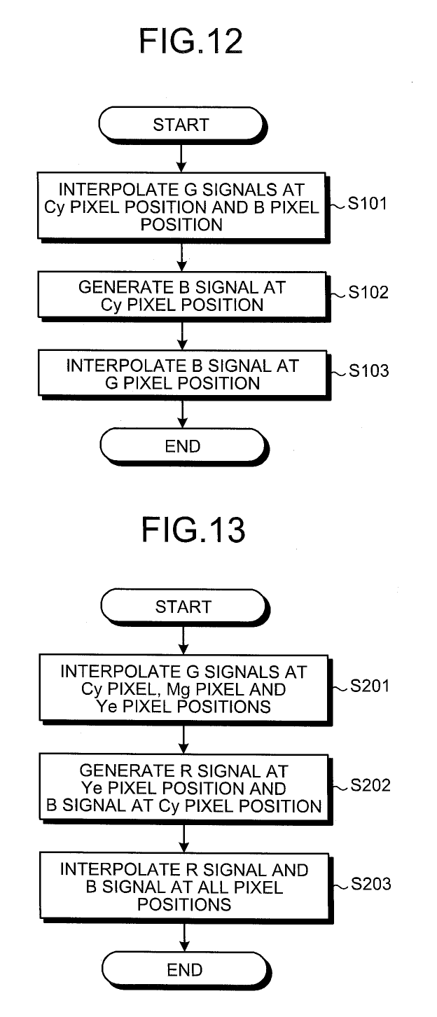

FIG. 12 is a flowchart illustrating the demosaicing processing in the NBI mode which is performed by the demosaicing processing unit of the endoscope apparatus according to the embodiment. First, the demosaicing processing unit 412 discriminates an interpolation direction using a pixel value generated by the G pixel, which is a non-selected pixel (pixel of a color component that is not the luminance component) and the luminance component of the white illumination light imaging, interpolates the G components in the B pixel (Mg pixel) as the selected pixel and the Cy pixel as the non-selected pixel based on the discriminated interpolation direction, and generates an image signal forming a single image in which each pixel has the pixel value or the interpolated value of the G component (step S101).

To be specific, the demosaicing processing unit 412 discriminates an edge direction from the existing G component (pixel value) as the interpolation direction, and executes the interpolation processing with respect to the B pixel and the Cy pixel serving as objects to be interpolated, along the interpolation direction. The demosaicing processing unit 412 calculates a signal value G(x,y) of the G component of a pixel to be interpolated in the coordinate (x,y) based on the discriminated edge direction using the following Formulas (1) to (6). The interpolation direction is discriminated from any of a vertical direction (Y direction), a horizontal direction (X direction), an obliquely downward direction, and an obliquely upward direction. In the discrimination of the edge direction, the up-and-down direction in the pixel arrangement illustrated in FIG. 3 is set as the vertical direction and the right-and-left direction thereof is set as the horizontal direction. In addition, the downward direction is set as a positive direction in the vertical direction, and a right direction is set as a positive direction in the right-and-left direction. In addition, when a pixel has no adjacent pixel, such as a pixel positioned at an outer edge, a signal value of a pixel positioned at a folded position is used.

Edge Direction: Vertical Direction

When a change of luminance in the horizontal direction is greater than each change of luminance in the other three directions, the demosaicing processing unit 412 discriminates the vertical direction as the edge direction and calculates the signal value G(x,y) using the following Formula (1). In Formula (1), a pixel to be interpolated is common between the B pixel and the Cy pixel.

$$G ( x , y ) = \frac{1}{2} \{ G ( x , y - 1 ) + G ( x , y + 1 ) \} \qquad ( 1 )$$

Edge Direction: Horizontal Direction

When a change of luminance in the vertical direction is greater than each change of luminance in the other three directions, the demosaicing processing unit 412 discriminates the horizontal direction as the edge direction and calculates the signal value G(x,y) using the following Formula (2).

$$G ( x , y ) = \frac{1}{2} \{ G ( x - 1 , y ) + G ( x + 1 , y ) \} \qquad ( 2 )$$

Edge Direction: Obliquely Downward Direction

When a change of luminance in the obliquely upward direction is greater than each change of luminance in the other three directions, the demosaicing processing unit 412 discriminates the obliquely downward direction as the edge direction. In this case, the demosaicing processing unit 412 calculates the signal value G(x,y) using the following Formula (3) if a pixel to be interpolated is the B pixel.

$$G ( x , y ) = \frac{1}{2} \{ Cy ( x - 1 , y - 1 ) + Cy ( x + 1 , y + 1 ) \} - B ( x , y ) \qquad ( 3 )$$

In addition, the demosaicing processing unit 412 discriminates the obliquely downward direction as the edge direction, and calculates the signal value G(x,y) using the following Formula (4) if a pixel to be interpolated is the Cy pixel.

$$G ( x , y ) = \frac{1}{4} \{ G ( x , y - 1 ) + G ( x , y + 1 ) + G ( x - 1 , y ) + G ( x + 1 , y ) \} \qquad ( 4 )$$

Edge Direction: Obliquely Upward Direction

When a change of luminance in the obliquely downward direction is greater than each change of luminance in the other three directions, the demosaicing processing unit 412 discriminates the obliquely upward direction as the edge direction. In this case, the demosaicing processing unit 412 calculates the signal value G(x,y) using the following Formula (5) if a pixel to be interpolated is the B pixel.

$$G ( x , y ) = \frac{1}{2} \{ Cy ( x + 1 , y - 1 ) + Cy ( x - 1 , y + 1 ) \} - B ( x , y ) \qquad ( 5 )$$

In addition, the demosaicing processing unit 412 discriminates the obliquely upward direction as the edge direction, and calculates the signal value G(x,y) using the following Formula (6) if a pixel to be interpolated is the Cy pixel.

$$G ( x , y ) = \frac{1}{4} \{ G ( x , y - 1 ) + G ( x , y + 1 ) + G ( x - 1 , y ) + G ( x + 1 , y ) \} \qquad ( 6 )$$

After performing the interpolation processing on the G signal using the above-described Formulas (1) to (6), the demosaicing processing unit 412 generates a signal value B(x,y) of the B component (the luminance component) of the Cy pixel (step S102). In the NBI mode, the beams of light of the narrow bands T_{B} and T_{G} are incident to the Cy pixel. Thus, it is possible to obtain the signal value B(x,y) corresponding to the light of the narrow band T_{B} by subtracting the signal value G(x,y) corresponding to the light of the narrow band T_{G} from an obtained signal value Cy(x,y). To be specific, the demosaicing processing unit 412 generates the signal value B(x,y) by subtracting the signal value (interpolated value) of the G component interpolated using the following Formula (7) from the signal value Cy(x,y) of the Cy component.

B(x,y)=Cy(x,y)−G(x,y) (7)

After generating the B signal in the Cy pixel using the above-described Formula (7), the demosaicing processing unit 412 interpolates the signal value B(x,y) of the B component (the luminance component) of the G pixel (step S103). To be specific, the demosaicing processing unit 412 interpolates the signal value B(x,y) of the B component (the luminance component) of the G pixel by performing calculation of the above-described Formulas (1) to (6) by substituting the signal value G(x,y) for the signal value B(x,y) and the signal value B(x,y) for the signal value G(x,y). Accordingly, the image signal having the signal value (the pixel value, the interpolated value or a signal value obtained by the subtraction) of the B component (the luminance component) and the G component is generated at least for the pixel forming the image.

Demosaicing Processing in WLI Mode

FIG. 13 is a flowchart illustrating the demosaicing processing in the WLI mode which is performed by the demosaicing processing unit of the endoscope apparatus according to the embodiment. First, the demosaicing processing unit 412 discriminates an interpolation direction using a pixel value generated by the G pixel, which is the selected pixel, interpolates the G components in the Cy pixel, the Mg pixel, and the Ye pixel as the non-selected pixels based on the discriminated interpolation direction, and generates an image signal forming a single image in which each pixel has the pixel value or the interpolated value of the G component (step S201).

To be specific, the demosaicing processing unit 412 discriminates an edge direction from the existing G component (pixel value) as the interpolation direction, and executes the interpolation processing with respect to the Cy pixel and the Mg pixel serving as objects to be interpolated, along the interpolation direction. The demosaicing processing unit 412 calculates a signal value G(x,y) of the G component of a pixel to be interpolated in the coordinate (x,y) based on the discriminated edge direction using the following Formulas (8) to (13). The interpolation direction in the WLI mode is discriminated from any of the vertical direction and the horizontal direction.

Edge Direction: Vertical Direction

When a change of luminance in the horizontal direction is greater than each change of luminance in the other three directions, the demosaicing processing unit 412 discriminates the vertical direction as the edge direction. In this case, the demosaicing processing unit 412 calculates the signal value G(x,y) using the following Formula (8) if a pixel to be interpolated is the Cy pixel.

$$G ( x , y ) = \frac{1}{2} \{ G ( x , y - 1 ) + G ( x , y + 1 ) \} \qquad ( 8 )$$

In addition, the demosaicing processing unit 412 discriminates the vertical direction as the edge direction, and calculates the signal value G(x,y) using the following Formula (9) if a pixel to be interpolated is the Mg pixel.

$$G ( x , y ) = \frac{1}{4} \{ Ye ( x , y - 1 ) + Ye ( x , y + 1 ) \} + \frac{1}{8} \{ Cy ( x - 1 , y - 1 ) + Cy ( x + 1 , y - 1 ) + Cy ( x - 1 , y + 1 ) + Cy ( x + 1 , y + 1 ) \} - \frac{1}{2} Mg ( x , y ) \qquad ( 9 )$$

In addition, the demosaicing processing unit 412 discriminates the vertical direction as the edge direction, and calculates the signal value G(x,y) using the following Formula (10) if a pixel to be interpolated is the Ye pixel.

$$G ( x , y ) = \frac{1}{4} \{ G ( x - 1 , y - 1 ) + G ( x + 1 , y - 1 ) + G ( x - 1 , y + 1 ) + G ( x + 1 , y + 1 ) \} \qquad ( 10 )$$

Edge Direction: Horizontal Direction

When a change of luminance in the vertical direction is greater than each change of luminance in the other three directions, the demosaicing processing unit 412 discriminates the horizontal direction as the edge direction. In this case, the demosaicing processing unit 412 calculates the signal value G(x,y) using the following Formula (11) if a pixel to be interpolated is the Cy pixel.

$$G ( x , y ) = \frac{1}{2} Cy ( x , y ) + \frac{1}{4} \{ Ye ( x - 1 , y ) + Ye ( x + 1 , y ) \} - \frac{1}{8} \{ Mg ( x - 1 , y - 1 ) + Mg ( x + 1 , y - 1 ) + Mg ( x - 1 , y + 1 ) + Mg ( x + 1 , y + 1 ) \} \qquad ( 11 )$$

In addition, the demosaicing processing unit 412 discriminates the horizontal direction as the edge direction, and calculates the signal value G(x,y) using the following Formula (12) if a pixel to be interpolated is the Mg pixel.

$$G ( x , y ) = \frac{1}{2} \{ G ( x - 1 , y ) + G ( x + 1 , y ) \} \qquad ( 12 )$$

In addition, the demosaicing processing unit 412 discriminates the horizontal direction as the edge direction, and calculates the signal value G(x,y) using the following Formula (13) if a pixel to be interpolated is the Ye pixel.

$$G ( x , y ) = \frac{1}{4} \{ G ( x - 1 , y - 1 ) + G ( x + 1 , y - 1 ) + G ( x - 1 , y + 1 ) + G ( x + 1 , y + 1 ) \} \qquad ( 13 )$$

After performing the interpolation processing on the G signal using the above-described Formulas (8) to (13), the demosaicing processing unit 412 generates a signal value R(x,y) of the R component of the Ye pixel and a signal value B(x,y) of the B component of the Cy pixel (step S202). In the WLI mode, the beams of light of the wavelength bands H_{R} and H_{G} are incident to the Ye pixel. Thus, it is possible to obtain the signal value R(x,y) corresponding to the light of the wavelength band H_{R} by subtracting the signal value G(x,y) corresponding to the light of the wavelength band H_{G} from an obtained signal value Ye(x,y). To be specific, the demosaicing processing unit 412 generates the signal value R(x,y) by subtracting the signal value (interpolated value) of the G component interpolated using the following Formula (14) from the signal value Ye(x,y) of the Ye component.

R(x,y)=Ye(x,y)−G(x,y) (14)

In addition, the beams of light of the wavelength bands H_{B} and H_{G} are incident to the Cy pixel. Thus, it is possible to obtain the signal value B(x,y) corresponding to the light of the wavelength band H_{B} by subtracting the signal value G(x,y) corresponding to the light of the wavelength band H_{G} from an obtained signal value Cy(x,y). To be specific, the demosaicing processing unit 412 generates the signal value B(x,y) by subtracting the signal value (interpolated value) of the G component interpolated using the following Formula (15) from the signal value Cy(x,y) of the Cy component.

B(x,y)=Cy(x,y)−G(x,y) (15)

After generating the R signal in the Ye pixel and the B signal in the Cy pixel using the above-described Formulas (14) and (15), the demosaicing processing unit 412 interpolates the signal value B(x,y) of the B component and the signal value R(x,y) of the R component for all pixel positions (step S203). To be specific, the demosaicing processing unit 412 performs the interpolation of the signal values R(x,y) and B(x,y) using the known bicubic interpolation. Accordingly, the image signal having the signal value (the pixel value, the interpolated value or a signal value obtained by the subtraction) of the G component (the luminance component), the B component, and the R component is generated at least for the pixel forming the image.

When each of the signal values of the color components is generated in the NBI mode and the WLI mode, the demosaicing processing unit 412 synchronizes the generated image signals of the luminance component and the color component, and generates a color image signal that includes a color image (synchronized image) to which the signal values of the RGB components or the GB components have been granted depending on each pixel. The color image generation processing unit 415c allocates the signals of the luminance component and the color component to each of RGB channels. A relationship between the channel and the signal in the imaging mode (WLI/NBI) is represented as below. In the embodiment, the signal of the luminance component is allocated to the G channel.

| | WLI Mode | NBI Mode |
|---|---|---|
| R Channel: | R Signal | G Signal |
| G Channel: | G Signal | B Signal |
| B Channel: | B Signal | B Signal |

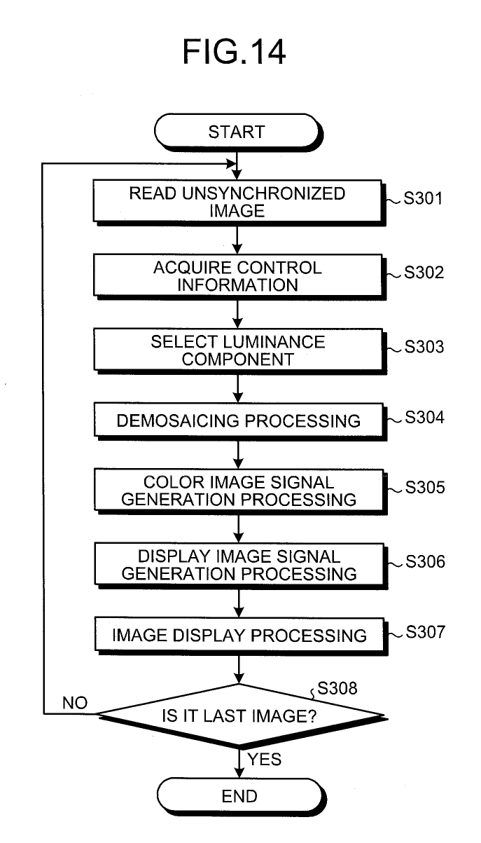

Next, reference will be made to the signal processing performed by the processor 4 having the above-described configuration with reference to the drawings. FIG. 14 is a flowchart illustrating the signal processing which is performed by the processor 4 of the endoscope apparatus 1 according to the embodiment. When acquiring an electric signal from the endoscope 2, the control unit 44 performs reading of an unsynchronized image included in the electric signal (step S301). The electric signal from the endoscope 2 is a signal that is generated by the image sensor 202 and includes unsynchronized image data that has been converted into a digital signal by the A/D converter 205.

After reading the unsynchronized image, the control unit 44 acquires the control information (for example, the information on the illumination light (imaging method) and the array information of the color filter 202a) with reference to the identification information storage unit 261 and outputs the acquired control information to the luminance component selector 411 (step S302).

The luminance component selector 411 determines any imaging method between the acquired white illumination light imaging method (WLI mode) and narrow band imaging method (NBI mode) that is used to generate the electric signal (unsynchronized image that has been read) based on the control information, and selects the selected pixel based on the determination (step S303). To be specific, the luminance component selector 411 selects the G component (the G pixel) as the luminance component (the selected pixel) when determining that the WLI mode is used, and selects the B component (pixel having the B component) as the luminance component (the selected pixel) when determining that the NBI mode is used. The luminance component selector 411 outputs the control signal relating to the selected luminance component to the demosaicing processing unit 412.

When an unsynchronized electric signal is input from the endoscope 2 (A/D converter 205), the demosaicing processing unit 412 performs the demosaicing processing based on the electric signal (step S304). To be specific, as in the above-described processing, the demosaicing processing unit 412 discriminates an interpolation direction in the pixel (pixel other than the G pixel) to be interpolated using the pixel value generated by the G pixel, which is the non-selected pixel in the case of the NBI mode, interpolates the G components in pixel positions other than the G pixel based on the discriminated interpolation direction, and generates an image signal forming a single image in which each pixel has the pixel value or the interpolated value of the luminance component. Thereafter, an image signal forming a single image including the pixel value or the interpolated value of the B component is generated for each of the color components based on the pixel value and the interpolated value of the G component and the pixel values of the pixels other than the pixel (selected pixel) of the luminance component. On the other hand, the demosaicing processing unit 412 discriminates an interpolation direction in the pixel (pixel other than the selected pixel) to be interpolated using the pixel value generated by the G pixel, which is the selected pixel in the case of the WLI mode, interpolates the G components in pixel positions other than the G pixel based on the discriminated interpolation direction, and generates an image signal forming a single image in which each pixel has the pixel value or the interpolated value of the luminance component. Thereafter, an image signal forming a single image including the pixel values or the interpolated values of the color components (the B component and the R component) other than the G component is generated for each of the color components based on the pixel value and the interpolated value of the G component and the pixel values of the pixels other than the selected pixel.

After generating the image signal for each of the color components, the demosaicing processing unit 412 generates a color image signal forming a color image using the respective image signals of the respective color components (step S305). The demosaicing processing unit 412 generates a color image signal using the image signals of the red component, the green component, and the blue component in the case of the WLI mode, and generates a color image signal using the image signals of the green component and the blue component in the case of the NBI mode.

After the color image signal is generated by the demosaicing processing unit 412, the display image generation processing unit 413 generates the display image signal for display by executing the gradation conversion, the magnification processing, and the like with respect to the color image signal (step S306). After executing the predetermined processing, the display image generation processing unit 413 outputs the processed signal to the display unit 5 as the display image signal.

When the display image signal is generated by the display image generation processing unit 413, image display processing is performed according to the display image signal (step S307). An image according to the display image signal is displayed on the display unit 5 through the image display processing.

After the display image generation processing unit 413 performs the process of generating the display image signal and the image display processing, the control unit 44 determines whether this image is the last image (step S308). The control unit 44 ends the process when a series of processing has been completed with respect to all images (Yes in step S308), and continues the same processing by causing the process to transition to the step S301 when there remains an unprocessed image (No in step S308).

Although, in the embodiment, each unit of the processor 4 is configured using hardware and each unit performs the processing, a CPU may perform the processing of each unit and the CPU executes a program to implement the above-described signal processing using software. For example, the signal processing may be also implemented by causing the CPU to execute the above-described software with respect to an image which is acquired, in advance, by an image sensor such as a capsule endoscope. In addition, a part of the processing performed by each unit may be configured using software. In this case, the CPU executes the signal processing according to the above-described flowchart.

According to the above-described embodiment, first, in the NBI mode, the signal of the non-luminance component (the G signal as the signal of the luminance component in the white illumination light imaging) is interpolated using the direction discrimination-type interpolation processing shown in Formulas (1) to (6) to generate the signal (B signal) of the luminance component at a Cy pixel position using the interpolated G signal and Formula (7). Finally, the direction discrimination-type interpolation processing is performed for the B signal. Since the interpolation processing for the B signal is performed after generating the B signal at the Cy pixel position, it is possible to improve the accuracy of the direction discrimination processing as well as to obtain a high-resolution image in any of the white light imaging method and the narrow band imaging method.

In addition, the complementary color filter is used and the filter array is given such that the G signal is present in all pixels in the up-and-down direction in the pixel to be interpolated of the G signal according to the above-described embodiment. Thus, it is possible to acquire the G signals in the checkerboard pattern in the NBI mode, and to function effectively for the direction discrimination-type interpolation processing.

In the above-described embodiment, the direction discrimination-type interpolation is performed. However, as long as the interpolation direction is set in advance or interpolation information is stored in the storage unit 43, the interpolation processing may be performed based on the setting or the interpolation information.

### First Modified Example of Embodiment

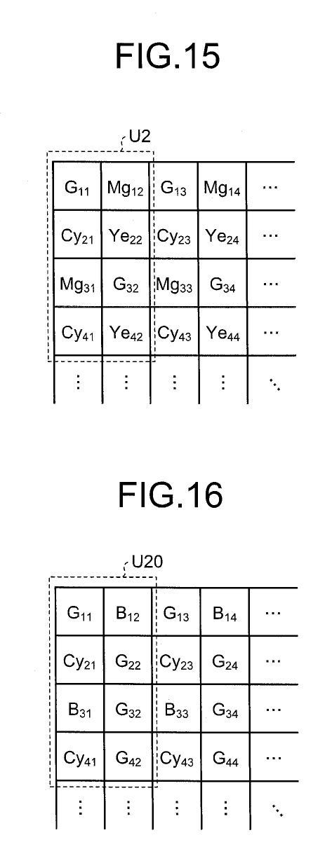

FIG. 15 is a schematic diagram illustrating a configuration of a color filter according to a first modified example of the embodiment. In the above-described embodiment, the color filter 202a is configured by arranging the filter units U1, formed using the four filters arrayed in a 2×2 matrix, to be arrayed in a matrix in accordance with the arrangement of the pixels Pᵢⱼ. In the first modified example, a color filter is configured by arranging filter units U2 arrayed in a matrix, using eight filters arrayed in a 2×4 matrix. The filter unit U2 is configured using a G filter, an Mg filter, a Cy filter, and a Ye filter as illustrated in FIG. 15. In the filter unit U2, the number of filters of each color is the same (two), and filters (same color filter) that pass light of a wavelength band of the same color are arranged not to be adjacent to each other in a row direction and a column direction.

Next, interpolation processing performed by the demosaicing processing unit 412 according to the first modified example will be described. Similarly to the above-described embodiment, the demosaicing processing unit 412 generates a color image signal by discriminating an interpolation direction from a correlation of color information (pixel values) of a plurality of pixels based on an unsynchronized imaging signal from the endoscope 2 (A/D converter 205) and performing interpolation based on the color information of the pixels arrayed in the discriminated interpolation direction. The demosaicing processing unit 412 performs different types of signal processing between the NBI mode and the WLI mode.

Demosaicing Processing in NBI Mode

FIG. 16 is a configuration of the color filter according to the first modified example of the embodiment and is a diagram for describing a function of the pixel in the NBI mode. Similarly to the above-described embodiment, an Mg pixel can be considered to be equivalent to a B pixel, and a Ye pixel can be considered to be equivalent to a G pixel. Accordingly, in the NBI mode, a complementary color filter array (filter unit U2) illustrated in FIG. 15 can be considered to be equivalent to a filter array (unitary unit U20) illustrated in FIG. 16. In the following demosaicing processing in the NBI mode, the filter array illustrated in FIG. 16 is employed in which the Mg pixel and the Ye pixel are regarded as the B pixel and the G pixel, respectively.

In the first modified example, the demosaicing processing unit 412 performs the demosaicing processing in accordance with the above-described flowchart illustrated in FIG. 12. First, the demosaicing processing unit 412 discriminates an interpolation direction using a pixel value generated by the G pixel, which is a non-selected pixel, interpolates G components in the B pixel (Mg pixel) as a selected pixel and a Cy pixel as the non-selected pixel based on the discriminated interpolation direction, and generates an image signal forming a single image in which each pixel has a pixel value or an interpolated value of the G component (step S101 in FIG. 12).

To be specific, the demosaicing processing unit 412 discriminates an edge direction from the existing G component (pixel value) as the interpolation direction, and executes the interpolation processing with respect to the B pixel and the Cy pixel serving as objects to be interpolated, along the interpolation direction. The demosaicing processing unit 412 calculates a signal value G(x,y) of the G component of a pixel to be interpolated in the coordinate (x,y) based on the discriminated edge direction using the following Formulas (16) to (20). The interpolation direction is discriminated from any of the vertical direction and the horizontal direction.

Edge Direction: Vertical Direction

When a change of luminance in the horizontal direction is greater than each change of luminance in the other three directions, the demosaicing processing unit 412 discriminates the vertical direction as the edge direction. In this case, the demosaicing processing unit 412 calculates the signal value G(x,y) using the following Formula (16) if a pixel to be interpolated is a B pixel and this B pixel is adjacent to a G pixel in the vertical direction (for example, B₁₂ in FIG. 16).

$$G ( x , y ) = \frac{1}{2} \{ G ( x , y - 1 ) + G ( x , y + 1 ) \} \qquad ( 16 )$$

In addition, the demosaicing processing unit 412 discriminates the vertical direction as the edge direction, and calculates the signal value G(x,y) using the following Formula (17) if a pixel to be interpolated is a B pixel and this B pixel is adjacent to a Cy pixel in the vertical direction (for example, B₃₁ in FIG. 16).

$$G ( x , y ) = \frac{1}{2} \{ Cy ( x , y - 1 ) + Cy ( x , y + 1 ) \} - B ( x , y ) \qquad ( 17 )$$

In addition, the demosaicing processing unit 412 discriminates the vertical direction as the edge direction, and calculates the signal value G(x,y) using the following Formula (18) if a pixel to be interpolated is a Cy pixel and this Cy pixel is adjacent to three G pixels in the upward direction (negative direction) and the obliquely downward direction (for example, Cy₂₁ in FIG. 16).

$$G ( x , y ) = \frac{1}{2} G ( x , y - 1 ) + \frac{1}{4} \{ G ( x + 1 , y + 1 ) + G ( x - 1 , y + 1 ) \} \qquad ( 18 )$$

In addition, the demosaicing processing unit 412 discriminates the vertical direction as the edge direction, and calculates the signal value G(x,y) using the following Formula (19) if a pixel to be interpolated is a Cy pixel and this Cy pixel is adjacent to three G pixels in the downward direction (positive direction) and the obliquely upward direction (for example, Cy₄₁ in FIG. 16).

$$G ( x , y ) = \frac{1}{2} G ( x , y + 1 ) + \frac{1}{4} \{ G ( x + 1 , y - 1 ) + G ( x - 1 , y - 1 ) \} \qquad ( 19 )$$

Edge Direction: Horizontal Direction

When a change of luminance in the vertical direction is greater than each change of luminance in the other three directions, the demosaicing processing unit 412 discriminates the horizontal direction as the edge direction and calculates the signal value G(x,y) using the following Formula (20). In Formula (20), the pixel to be interpolated is common between the B pixel and the Cy pixel.

$$G ( x , y ) = \frac{1}{2} \{ G ( x - 1 , y ) + G ( x + 1 , y ) \} \qquad ( 20 )$$

After performing the interpolation processing on the G signal using the above-described Formulas (16) to (20), the demosaicing processing unit 412 generates a signal value B(x,y) of the B component (luminance component) of the Cy pixel using Formula (7) (step S102 in FIG. 12).

After generating the B signal in the Cy pixel using the above-described Formula (7), the demosaicing processing unit 412 interpolates the signal value B(x,y) of the B component (the luminance component) of the G pixel (step S103 in FIG. 12).

Demosaicing Processing in WLI Mode

In this modified example, the demosaicing processing unit 412 performs the demosaicing processing in accordance with the above-described flowchart illustrated in FIG. 13. First, the demosaicing processing unit 412 discriminates an interpolation direction using a pixel value generated by the G pixel, which is the selected pixel, interpolates the G components in the Cy pixel, the Mg pixel, and the Ye pixel as the non-selected pixels based on the discriminated interpolation direction, and generates an image signal forming a single image in which each pixel has the pixel value or the interpolated value of the G component (step S201 in FIG. 13).

To be specific, the demosaicing processing unit 412 discriminates an edge direction from the existing G component (pixel value) as the interpolation direction, and executes the interpolation processing with respect to the Cy pixel and the Mg pixel serving as objects to be interpolated, along the interpolation direction. The demosaicing processing unit 412 calculates a signal value G(x,y) of the G component of a pixel to be interpolated in the coordinate (x,y) based on the discriminated edge direction using the following Formulas (21) to (29). The interpolation direction in the WLI mode is discriminated from any of the vertical direction and the horizontal direction.

Edge Direction: Vertical Direction

When a change of luminance in the horizontal direction is greater than each change of luminance in the other three directions, the demosaicing processing unit 412 discriminates the vertical direction as the edge direction. In this case, the demosaicing processing unit 412 calculates the signal value G(x,y) using the following Formula (21) if a pixel to be interpolated is a Cy pixel or a Ye pixel and this pixel is adjacent to three G pixels in the upward direction (negative direction) and the obliquely downward direction (for example, Cy₂₁ or Ye₄₂ in FIG. 15).

$$G ( x , y ) = \frac{1}{2} G ( x , y - 1 ) + \frac{1}{4} \{ G ( x - 1 , y + 1 ) + G ( x + 1 , y + 1 ) \} \qquad ( 21 )$$

In addition, the demosaicing processing unit 412 discriminates the vertical direction as the edge direction, and calculates the signal value G(x,y) using the following Formula (22) if a pixel to be interpolated is a Cy pixel or a Ye pixel and this pixel is adjacent to three G pixels in the downward direction (positive direction) and the obliquely upward direction (for example, Cy₄₁ or Ye₂₂ in FIG. 15).

$$G ( x , y ) = \frac{1}{2} G ( x , y + 1 ) + \frac{1}{4} \{ G ( x - 1 , y - 1 ) + G ( x + 1 , y - 1 ) \} \qquad ( 22 )$$

In addition, the demosaicing processing unit 412 discriminates the vertical direction as the edge direction, and calculates the signal value G(x,y) using the following Formula (23) if a pixel to be interpolated is an Mg pixel, and this Mg pixel is adjacent to a Ye pixel in the vertical direction and adjacent to four Cy pixels in the oblique direction (for example, Mg₁₂ in FIG. 15).

$$G ( x , y ) = \frac{1}{4} \{ Ye ( x , y - 1 ) + Ye ( x , y + 1 ) \} + \frac{1}{8} \{ Cy ( x - 1 , y - 1 ) + Cy ( x + 1 , y - 1 ) + Cy ( x - 1 , y + 1 ) + Cy ( x + 1 , y + 1 ) \} - \frac{1}{2} Mg ( x , y ) \qquad ( 23 )$$

In addition, the demosaicing processing unit 412 discriminates the vertical direction as the edge direction, and calculates the signal value G(x,y) using the following Formula (24) if a pixel to be interpolated is an Mg pixel, and this Mg pixel is adjacent to a Cy pixel in the vertical direction and adjacent to four Ye pixels in the oblique direction (for example, Mg₃₁ in FIG. 15).

$$G ( x , y ) = \frac{1}{4} \{ Cy ( x , y - 1 ) + Cy ( x , y + 1 ) \} + \frac{1}{8} \{ Ye ( x - 1 , y - 1 ) + Ye ( x + 1 , y - 1 ) + Ye ( x - 1 , y + 1 ) + Ye ( x + 1 , y + 1 ) \} - \frac{1}{2} Mg ( x , y ) \qquad ( 24 )$$

Edge Direction: Horizontal Direction

When a change of luminance in the vertical direction is greater than each change of luminance in the other three directions, the demosaicing processing unit 412 discriminates the horizontal direction as the edge direction. In this case, the demosaicing processing unit 412 calculates the signal value G(x,y) using the following Formula (25) if a pixel to be interpolated is a Cy pixel and this Cy pixel is adjacent to a Ye pixel in the vertical direction and adjacent to Mg pixels in the downward direction, the left obliquely upward direction, and the right obliquely upward direction (for example, Cy₂₁ in FIG. 15).

$$G ( x , y ) = \frac{1}{2} Cy ( x , y ) + \frac{1}{4} \{ Ye ( x - 1 , y ) + Ye ( x + 1 , y ) \} - \frac{1}{6} \{ Mg ( x - 1 , y - 1 ) + Mg ( x + 1 , y - 1 ) + Mg ( x , y + 1 ) \} \qquad ( 25 )$$

In addition, the demosaicing processing unit 412 discriminates the horizontal direction as the edge direction, and calculates the signal value G(x,y) using the following Formula (26) if a pixel to be interpolated is a Cy pixel and this Cy pixel is adjacent to a Ye pixel in the vertical direction and adjacent to Mg pixels in the upward direction, the left obliquely downward direction, and the right obliquely downward direction (for example, Cy₄₁ in FIG. 15).

$$G ( x , y ) = \frac{1}{2} Cy ( x , y ) + \frac{1}{4} \{ Ye ( x - 1 , y ) + Ye ( x + 1 , y ) \} - \frac{1}{6} \{ Mg ( x - 1 , y + 1 ) + Mg ( x + 1 , y + 1 ) + Mg ( x , y - 1 ) \} \qquad ( 26 )$$

In addition, the demosaicing processing unit 412 discriminates the horizontal direction as the edge direction, and calculates the signal value G(x,y) using the following Formula (27) if a pixel to be interpolated is a Ye pixel and this Ye pixel is adjacent to a Cy pixel in the vertical direction and adjacent to Mg pixels in the upward direction, the left obliquely downward direction, and the right obliquely downward direction (for example, Ye₂₂ in FIG. 15).

$$G ( x , y ) = \frac{1}{2} Ye ( x , y ) + \frac{1}{4} \{ Cy ( x - 1 , y ) + Cy ( x + 1 , y ) \} - \frac{1}{6} \{ Mg ( x - 1 , y + 1 ) + Mg ( x + 1 , y + 1 ) + Mg ( x , y - 1 ) \} \qquad ( 27 )$$

In addition, the demosaicing processing unit 412 discriminates the horizontal direction as the edge direction, and calculates the signal value G(x,y) using the following Formula (28) if a pixel to be interpolated is a Ye pixel and this Ye pixel is adjacent to a Cy pixel in the vertical direction and adjacent to Mg pixels in the downward direction, the left obliquely upward direction, and the right obliquely upward direction (for example, Ye₄₂ in FIG. 15).

$$G ( x , y ) = \frac{1}{2} Ye ( x , y ) + \frac{1}{4} \{ Cy ( x - 1 , y ) + Cy ( x + 1 , y ) \} - \frac{1}{6} \{ Mg ( x - 1 , y - 1 ) + Mg ( x + 1 , y - 1 ) + Mg ( x , y + 1 ) \} \qquad ( 28 )$$

In addition, the demosaicing processing unit 412 discriminates the horizontal direction as the edge direction, and calculates the signal value G(x,y) using the following Formula (29) if a pixel to be interpolated is the Mg pixel.

$$G ( x , y ) = \frac{1}{2} \{ G ( x - 1 , y ) + G ( x + 1 , y ) \} \qquad ( 29 )$$

After performing the interpolation processing on the G signal using the above-described Formulas (21) to (29), the demosaicing processing unit 412 generates a signal value R(x,y) of the R component of the Ye pixel and a signal value B(x,y) of the B component of the Cy pixel using Formulas (14) and (15) (step S202 in FIG. 13).

After generating the R signal in the Ye pixel and the B signal in the Cy pixel using the above-described Formulas (14) and (15), the demosaicing processing unit 412 interpolates the signal value B(x,y) of the B component and the signal value R(x,y) of the R component for all pixel positions (step S203 in FIG. 13).

In the above-described first modified example, similarly to the above-described embodiment, first, in the NBI mode, the signal of the non-luminance component (the G signal) is interpolated using the direction discrimination-type interpolation processing shown in Formulas (16) to (20) to generate the signal (B signal) of the luminance component at a Cy pixel position using the interpolated G signal and Formula (7). Finally, the direction discrimination-type interpolation processing is performed for the B signal. Since the interpolation processing for the B signal is performed after generating the B signal at the Cy pixel position, it is possible to improve the accuracy of the direction discrimination processing as well as to obtain a high-resolution image in any of the white light imaging method and the narrow band imaging method.

### Second Modified Example of Embodiment

In the above-described embodiment, the interpolation processing is performed by discriminating the interpolation direction using Formulas (8) to (13) in the WLI mode. In the second modified example, YCbCr is generated by performing addition or subtraction processing on an adjacent signal of a different color, and further, a color image is generated using conversion from YCbCr into RGB defined in the international standard ITU-R BT.709.

To be specific, the demosaicing processing unit 412 generates a signal value Y(x,y) of a luminance component, signal values Cb(x,y) and Cr(x,y) of color difference components between color components and the luminance component using the following Formulas (30) to (32).

$$\begin{matrix}Y ( x , y ) = \frac{1}{4} \{ G ( x , y ) + Mg ( x + 1 , y ) + Cy ( x , y + 1 ) + Ye ( x + 1 , y + 1 ) \} & ( 30 ) \\ Cb ( x , y ) = Mg ( x + 1 , y ) + Cy ( x , y + 1 ) - \{ G ( x , y ) + Ye ( x + 1 , y + 1 ) \} & ( 31 ) \\ Cr ( x , y ) = Mg ( x + 1 , y ) + Ye ( x + 1 , y + 1 ) - \{ G ( x , y ) + Cy ( x , y + 1 ) \} & ( 32 )\end{matrix}$$

Thereafter, when generating the signal values Y(x,y), Cb(x,y) and Cr(x,y), the demosaicing processing unit 412 performs the conversion from YCbCr into RGB using the following Formula (33) to generate signal values R(x,y), G(x,y) and B(x,y) of an R component, a G component, a B component, and generates a color image based on the obtained signal values of the RGB components.

R(x,y)=Y(x,y)+1.5748×Cr(x,y)

G(x,y)=Y(x,y)−0.1873×Cb(x,y)−0.4681×Cr(x,y)

B(x,y)=Y(x,y)+1.8556×Cb(x,y) (33)

### Third Modified Example of Embodiment

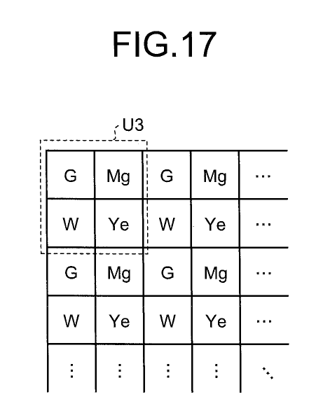

In addition, the color filter array is not limited to the above-described embodiment and the like. FIG. 17 is a schematic diagram illustrating a configuration of a color filter according to a third modified example of the embodiment. In the above-described embodiment, the filter unit U1 is configured using the green filter (G filter), the magenta filter (Mg filter), the cyan filter (Cy filter), and the yellow filter (Ye filter). In the third modified example, a filter unit U3 includes a white filter (W filter), which passes light of the wavelength band H_{G}, the wavelength band H_{B} and the wavelength band H_{R} instead of the Cy filter of the filter unit U1. In the third modified example, a pixel of a luminance component in an NBI mode is a W pixel, and a pixel of a luminance component in a WLI mode is a G pixel.

Since the NBI observation is used as the narrow band illumination light imaging even in this third modified example, the W filter corresponds to the first filter that passes light of the wavelength bands (wavelength bands H_{B} and H_{G}) of the respective luminance components in the white illumination light imaging and the narrow band illumination light imaging, the G filter and the Ye filter correspond to the second filters that pass light of the wavelength band (wavelength band H_{G}) of the luminance component (green component) in the white illumination light imaging and block light of the wavelength band of the luminance component (blue component) in the narrow band illumination light imaging, and the Mg filter corresponds to the third filter that passes light of the wavelength band (the wavelength band H_{B}) of the luminance component in the narrow band illumination light imaging and blocks light of the wavelength band of the luminance component in the white illumination light imaging.

Demosaicing Processing in NBI Mode

In the NBI mode, it is possible to consider the filter array (filter unit U10) illustrated in FIG. 11. Thus, it is possible to apply the demosaicing processing according to the above-described embodiment in the NBI mode.

Demosaicing Processing in WLI Mode

In this third modified example, the demosaicing processing is performed with reference to the flowchart illustrated in FIG. 13. First, the demosaicing processing unit 412 discriminates an interpolation direction using a pixel value generated by the G pixel as a selected pixel, interpolates the G component in the W pixel, the Mg pixel, and the Ye pixel as non-selected pixels based on the discriminated interpolation direction, and generates an image signal forming a single image in which each pixel has a pixel value or an interpolated value of the G component (step S201).

To be specific, the demosaicing processing unit 412 discriminates an edge direction from the existing G component (pixel value) as the interpolation direction, and executes the interpolation processing with respect to the W pixel and the Mg pixel serving as objects to be interpolated, along the interpolation direction. The demosaicing processing unit 412 calculates a signal value G(x,y) of the G component of a pixel to be interpolated in the coordinate (x,y) based on the discriminated edge direction.

Edge Direction: Vertical Direction

When a change of luminance in the horizontal direction is greater than each change of luminance in the other three directions, the demosaicing processing unit 412 discriminates the vertical direction as the edge direction. In this case, the demosaicing processing unit 412 calculates the signal value G(x,y) using the above-described Formula (8) if a pixel to be interpolated is the W pixel.

In addition, the demosaicing processing unit 412 discriminates the vertical direction as the edge direction, and calculates the signal value G(x,y) using the following Formula (34) if a pixel to be interpolated is the Mg pixel.

$$G ( x , y ) = \frac{1}{4} \{ W ( x - 1 , y - 1 ) + W ( x + 1 , y - 1 ) + W ( x - 1 , y + 1 ) + W ( x + 1 , y + 1 ) \} - Mg ( x , y ) \qquad ( 34 )$$

In addition, the demosaicing processing unit 412 discriminates the vertical direction as the edge direction, and calculates the signal value G(x,y) using the above-described Formula (10) if a pixel to be interpolated is the Ye pixel.

Edge Direction: Horizontal Direction

When a change of luminance in the vertical direction is greater than each change of luminance in the other three directions, the demosaicing processing unit 412 discriminates the horizontal direction as the edge direction. In this case, the demosaicing processing unit 412 calculates the signal value G(x,y) using the following Formula (35) if a pixel to be interpolated is the W pixel.

$$G ( x , y ) = W ( x , y ) - \frac{1}{4} \{ Mg ( x - 1 , y - 1 ) + Mg ( x + 1 , y - 1 ) + Mg ( x - 1 , y + 1 ) + Mg ( x + 1 , y + 1 ) \} \qquad ( 35 )$$

In addition, the demosaicing processing unit 412 discriminates the horizontal direction as the edge direction, and calculates the signal value G(x,y) using the above-described Formula (12) if a pixel to be interpolated is the Mg pixel.

In addition, the demosaicing processing unit 412 discriminates the horizontal direction as the edge direction, and calculates the signal value G(x,y) using the above-described Formula (13) if a pixel to be interpolated is the Ye pixel.

After performing the interpolation processing on the G signal using the above-described formula, the demosaicing processing unit 412 generates a signal value R(x,y) of the R component of the Ye pixel and a signal value B(x,y) of the B component of the W pixel (step S202). In the WLI mode, the beams of light of the wavelength bands H_{R} and H_{G} are incident to the Ye pixel. Thus, it is possible to obtain the signal value R(x,y) corresponding to the light of the wavelength band H_{R} by subtracting the signal value G(x,y) corresponding to the light of the wavelength band H_{G} from an obtained signal value Ye(x,y). To be specific, the demosaicing processing unit 412 generates the signal value R(x,y) by subtracting the signal value (interpolated value) of the G component interpolated using the above-described Formula (14) from the signal value Ye(x,y) of the Ye component.

In addition, the beams of light of the wavelength bands H_{B}, H_{G} H_{R} are incident to the W pixel. Thus, it is possible to obtain the signal value B(x,y) corresponding to the light of the wavelength band H_{B} by subtracting the signal value G(x,y) corresponding to the light of the wavelength band H_{G} from an obtained signal value W(x,y). To be specific, the demosaicing processing unit 412 generates the signal value B(x,y) by subtracting the signal value (interpolated value) of the G component interpolated using the following Formula (36) from the signal value W(x,y) of the W component.

B(x,y)=W(x,y)−G(x,y)−R(x,y) (36)

The signal value R(x,y) corresponding to the light of the wavelength band H_{R} in the W pixel can be obtained by known bicubic interpolation using the interpolated signal values R(x,y) of the Mg pixel and the Ye pixel. The signal value B(x,y) can be generated by subtracting the signal value R(x,y) obtained by the bicubic interpolation and the signal value (including the interpolated value) G(x,y) of the G component from the signal value W (x,y).

After generating the R signal in the Ye pixel and the B signal in the W pixel using the above-described formula, the demosaicing processing unit 412 interpolates the signal value B(x,y) of the B component and the signal value R(x,y) of the R component for all pixel positions (step S203). Accordingly, the image signal having the signal value (the pixel value, the interpolated value or a signal value obtained by the subtraction) of the G component (the luminance component), the B component, and the R component is generated at least for the pixel forming the image.

The above-described filter unit in the color filter 202a according to the above-described embodiment includes the filters arranged in 2×2 matrix or 2×4 matrix. However, another matrix arrangement may be employed.

In the above-described embodiment, the color filter 202a including the plurality of filters each of which passes light of the predetermined wavelength band is provided on the light receiving surface of the image sensor 202. However, the filters may be individually provided for each pixel of the image sensor 202.

The endoscope apparatus 1 according to the above-described embodiment performs switching of the illumination light, emitted from the illumination unit 31 depending on the insertion or removal of the switching filter 31c, to any light between the white illumination light and the narrow band illumination light, with respect to the white light emitted from the single light source 31a. However, two light sources that emit the white illumination light and the narrow band illumination light, respectively, may be switched to emit any of the white illumination light and the narrow band illumination light. When the two light sources are switched to emit any of the white illumination light and the narrow band illumination light, the invention can be also applied to a capsule endoscope which includes, for example, a light source unit, a color filter, and an image sensor and is introduced into a subject.

The A/D converter 205 is provided at the distal end portion 24 in the endoscope apparatus 1 according to the above-described embodiment. However, the A/D converter 205 may be provided in the processor 4. In addition, the configuration relating to the image processing may be provided in the endoscope 2, a connector that connects the endoscope 2 and the processor 4, the operating unit 22, and the like. In addition, the endoscope 2 connected to the processor 4 is identified using the identification information or the like stored in the identification information storage unit 261 in the above-described endoscope apparatus 1. However, an identification unit may be provided in a connecting part (connector) between the processor 4 and the endoscope 2. For example, a pin for identification (the identification unit) is provided on the endoscope 2 side to identify the endoscope 2 connected to the processor 4.

Although, in the above-described embodiment, a motion vector is detected after performing synchronization on the luminance component by the demosaicing processing unit 412, the present invention is not limited thereto. A configuration of detecting a motion vector using a unsynchronized luminance signal (pixel value) may be used as another method. In this case, the pixel value is not obtained from a pixel (non-selected pixel) other than a selected pixel at the time of performing matching between the same color pixels, and thus, it is possible to reduce operating cost required for block matching although an interval of the matching is limited. Herein, the motion vector is detected only from the selected pixel, and thus, it is necessary to interpolate the motion vector in the non-selected pixel. The known bicubic interpolation may be used as the interpolation processing at this time.

According to some embodiments, it is possible to obtain a high-resolution image in any of the white light imaging method and the narrow band imaging method.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope apparatus for performing white illumination light imaging and narrow band illumination light imaging, the endoscope apparatus comprising:
a light source unit configured to emit one of white illumination light and narrow band illumination light, the white illumination light including red wavelength band light, green wavelength band light, and blue wavelength band light, the narrow band illumination light including light of two narrow bands included in a wavelength band of a luminance component in the white illumination light imaging and a wavelength band of a luminance component in the narrow band illumination light imaging;
an image sensor including a plurality of pixels arranged in matrix and configured to perform photoelectric conversion on light received by each of the plurality of pixels to generate an electric signal;
a color filter arranged on a light receiving surface of the image sensor and formed by arraying a plurality of filter units, each of the plurality of filter units including a first filter, a second filter, and a third filter, the first filter being configured to pass light of the wavelength band of the luminance component in the white illumination light imaging and the wavelength band of the luminance component in the narrow band illumination light imaging, the second filter being configured to pass light of the wavelength band of the luminance component in the white illumination light imaging, and the third filter being configured to pass light of the wavelength band of the luminance component in the narrow band illumination light imaging; and
a demosaicing processing unit configured to:
generate a color image signal including a plurality of color components based on the luminance component in the white illumination light imaging when the light source unit emits the white illumination light; and
perform interpolation for a pixel value of the luminance component in the white illumination light imaging at a position of a pixel corresponding to the first filter using a pixel value of a pixel corresponding to the second filter when the light source unit emits the narrow band illumination light, and then, perform interpolation for a pixel value of the luminance component in the narrow band illumination light imaging at the position of the pixel corresponding to the first filter based on a pixel value of the pixel corresponding to the first filter and the pixel value of the luminance component in the white illumination light imaging obtained by the interpolation, thereby generating a color image signal including a plurality of color components.

2. The endoscope apparatus according to claim 1, wherein
the demosaicing processing unit is configured to perform the interpolation for the pixel value of the luminance component in the narrow band illumination light imaging at the position of the pixel corresponding to the first filter by calculating a difference between the pixel value of the pixel corresponding to the first filter and the pixel value of the luminance component in the white illumination light imaging obtained by the interpolation.

3. The endoscope apparatus according to claim 1, wherein
the color filter is arranged on the light receiving surface of the image sensor and is formed by arraying the plurality of filter units, each of the plurality of filter units including a green filter for passing the green wavelength band light, a cyan filter for passing the green wavelength band light and the blue wavelength band light, a magenta filter for passing the red wavelength band light and the blue wavelength band light, and a yellow filter for passing the red wavelength band light and the green wavelength band light.

4. The endoscope apparatus according to claim 1, wherein
the color filter is arranged on the light receiving surface of the image sensor and is formed by arraying the plurality of filter units, each of the plurality of filter units including a green filter for passing the green wavelength band light, a white filter for passing the red wavelength band light, the green wavelength band light and the blue wavelength band, a magenta filter for passing the red wavelength band light and the blue wavelength band light, and a yellow filter for passing the red wavelength band light and the green wavelength band light.

5. The endoscope apparatus according to claim 1, wherein
the light source unit is configured to emit the narrow band illumination light including the light of the two narrow bands included in blue and green wavelength bands, respectively, during the narrow band illumination light imaging.

6. The endoscope apparatus according to claim 5, wherein
the luminance component in the white illumination light imaging is a green component, and the luminance component in the narrow band illumination light imaging is a blue component, and
when the light source unit emits the narrow band illumination light, the demosaicing processing unit is configured to generate a pixel value of the blue component after performing the interpolation for a pixel value of the green component.

7. The endoscope apparatus according to claim 1, wherein
when the light source unit emits the white illumination light, the demosaicing processing unit is configured to perform addition and subtraction processing using signal values of different color components of adjacent pixels, thereby generating the color image signal including the plurality of color components.

8. The endoscope apparatus according to claim 1, wherein
the demosaicing processing unit is configured to discriminate an interpolation direction from a signal value of a same color component of surrounding pixels, and perform the interpolation along the discriminated interpolation direction.

9. The endoscope apparatus according to claim 1, wherein
the first and third filters for passing the light of the wavelength band of the luminance component in the narrow band illumination light imaging, are arranged in a checkerboard pattern in the color filter.
